(19) United States
(12) Patent Application Publication
(10) Pub. No.: US 2024/0423721 A1
(43) Pub. Date: Dec. 26, 2024

(54) METHODS OF PLANNING A ROBOT-IMPLEMENTED DENTAL PROCEDURE

(71) Applicant: NEOCIS INC., Miami, FL (US)

(72) Inventors: Uday N. Reebye, Durham, NC (US); Alon Mozes, Miami Beach, FL (US); Dennis Moses, Hollywood, FL (US); James Scott Tieman, Coral Gables, FL (US); Alexandra Bellettre, Salt Lake City, UT (US); Carter Jonah Randall, Miami, FL (US); Pranav Vikas Keni, Miami, FL (US); Kevin Christopher Chazotte, Miami Beach, FL (US)

(21) Appl. No.: 18/708,176

(22) PCT Filed: Nov. 14, 2022
(86) PCT No.: PCT/IB2022/060945

§ 371 Date: May 7, 2024

Related U.S. Application Data
(60) Provisional application No. 63/279,454, filed on Nov. 15, 2021.

(51) Int. Cl. A61B 34/10; A61B 34/30; A61C 1/08

(52) U.S. Cl. CPC A61B 34/10; A61B 34/30; A61C 1/084; A61B 2034/107; A61B 2034/305

(57) ABSTRACT

A method of forming a procedure plan includes arranging a tracking arm distal end in communication with a reference location (RL) about a site or an object received thereat (S/O), the RL disposed in relation to the tracking arm in a 3D space. Physical points about the S/O are each contacted with an end effector of a procedure tool engaged with a robot arm disposed in known relation to the tracking arm, and locations thereof concurrently determined in the 3D space. A reference frame (RF) is formed in the 3D space, relative to the RL, from the locations of the physical points, and includes a location of the S/O within the RF. A plan is formed for an end effector procedure on the S/O, within the RF and relative to the RL, including a route traversed by the end effector to and from the S/O and during the procedure.

### BACKGROUND

### Field of the Disclosure

The present disclosure relates to dental robot systems and, more particularly to methods of planning and executing a robot-implemented dental procedure.

### Description of Related Art

Certain dental or maxillofacial procedures, particularly robot-assisted procedures, generally require one or more forms of imaging of the jaws or maxillofacial bone structure in order to assess the bone structure and form an effective plan of the procedure. For example, one robot-assisted dental/maxillofacial procedure first requires that a splint device with fiducial markers be securely attached to the patient, generally in proximity to the intended site for the procedure. The patient is then subjected to a 2D or 3D imaging procedure (e.g., an X-ray scan or CT scan) such that the splint device, the fiducial markers, and the site appear in the resulting image(s). A plan for the procedure is then formed from the captured images, via appropriate software, with respect to a 3D virtual representation of the site, and in relation to the imaged fiducial markers. Once the plan for the procedure is formed via the software, a tracking system for the robot is attached to the patient via the splint device, in a known relationship with the fiducial markers. The tracking system assists in guiding the robot according to the software plan in relation to the fiducial markers. Since the location of the fiducial markers is known and monitored by the tracking system, the guidance of the robot according to the software plan is updated in real time to account for movement of the patient (i.e., the software plan is executed in relation to the fiducial markers, and the real-time location of the fiducial markers is tracked and provided by the tracking system).

However, in such a robot-assisted procedure, the imaging procedure(s) required to form the 3D virtual representation of the site often represents added time and expense, as well as potential inconvenience for the patient. The splint device must also remain affixed to the patient from before the imaging procedure(s) through formation of the software plan and until completion of the actual procedure in order to ensure that the fiducial markers remain fixed as a reliable reference point. If the splint device is removable between the imaging stage and the conduct of the procedure, then significant care must be taken to ensure that the splint device can be re-affixed in the same manner and in the same positon upon which the location of the fiducial markers was originally established.

As such, there exists a need for a method of planning and executing a procedure (e.g., a dental or maxillofacial procedure), and particularly a robot-assisted or robot-implemented procedure, that can be accomplished, without requiring one or more forms of imaging of the site (e.g., jaws or maxillofacial bone structure). It would also be desirable, in some instances, for such a method to be accomplished while the site is established in a reference frame known to the robot system, such that the planning and conducting of the procedure on the site can be conducted in a compact time frame and without requiring the site to be subject to a separate imaging system beforehand, at added time and expense. Moreover, such a method should be ergonomically friendly, expedient, and convenient for the dental professional, for example, by minimizing the steps necessary to effectuate the plan and obviating the need for interchanging instruments between planning and conducting the procedure.

### SUMMARY

The above and other needs are met by aspects of the present disclosure which, in one aspect, provides a method of forming a plan for performing a procedure, wherein such a method comprises arranging a distal end of a tracking arm in communication with a reference location at or adjacent to a site or an object received at the site, with the reference location being disposed in a relation to a proximal end of the tracking arm in a three-dimensional space. Each of a plurality of physical points at or adjacent to the site or the object are contacted with a distal end of an end effector of a procedure tool engaged with a distal end of a robot arm, wherein a proximal end of the robot arm is disposed in a known relation to the proximal end of the tracking arm. A location of the distal end of the end effector in the three-dimensional space is determined concurrently with contacting each of the physical points with the distal end of the end effector. A reference frame is formed in the three-dimensional space, in relation to the reference location, from the locations of the physical points, and to include a location of the site or the object within the reference frame. A plan is formed for a procedure to be performed by the end effector on the site or the object, within the reference frame and relative to the reference location, wherein the procedure includes a route traversed by the end effector to and from the site or the object and during the procedure performed on the site or the object by the end effector, and a trajectory of the end effector during the procedure.

Another aspect of the present disclosure provides a method of performing a procedure, wherein such a method comprises arranging a distal end of a tracking arm in communication with a reference location at or adjacent to a site or an object received at the site, with the reference location being disposed in a three-dimensional space relative to a proximal end of the tracking arm. Each of a plurality of physical points at or adjacent to the site or the object is contacted with a distal end of an end effector of a procedure tool engaged with a distal end of a robot arm, wherein a proximal end of the robot arm is disposed in a known relation to the proximal end of the tracking arm. A location of the distal end of the end effector in the three-dimensional space is determined concurrently with contacting each of the physical points with the distal end of the end effector. A reference frame is formed in the three-dimensional space, in relation to the reference location, from the locations of the physical points, and to include a location of the site or the object within the reference frame. A plan is formed for a procedure to be performed by the end effector on the site or the object, within the reference frame and relative to the reference location, wherein the procedure includes a route traversed by the end effector to and from the site or the object and during the procedure performed on the site or the object by the end effector, and a trajectory of the end effector during the procedure. The procedure on the site or the object is performed according to the plan, including traversing the route, using the end effector of the procedure tool engaged with the distal end of the robot arm.

Yet another aspect of the present disclosure provides a method of performing a procedure, wherein such a method comprises arranging a distal end of a tracking arm in communication with a reference location at or adjacent to a site or an object received at the site, wherein the reference location is disposed in a three-dimensional space relative to a proximal end of the tracking arm. A physical point at the site or on the object is contacted with a distal end of an end effector of a procedure tool engaged with a distal end of a robot arm, wherein a proximal end of the robot arm is disposed in a known relation to the proximal end of the tracking arm. A location of the distal end of the end effector in the three-dimensional space is determined concurrently with contacting the physical point with the distal end of the end effector. A trajectory of the end effector is established, relative to the physical point at the site or on the object, for performing a procedure at the site or on the object. A plan is formed for the procedure to be performed by the end effector at the location of the physical point at the site or on the object, wherein the procedure includes a route traversed by the end effector to and from the site or the object and during the procedure performed by the end effector at the location and in the established trajectory at the physical point at the site or on the object. The procedure at the site or on the object is performed according to the plan, including traversing the route, using the end effector of the procedure tool engaged with the distal end of the robot arm.

Still another aspect of the present disclosure provides a method of performing a procedure. Such a method comprises arranging a distal end of a tracking arm in communication with a reference location at or adjacent to a site or an object received at the site, wherein the reference location is disposed in a three-dimensional space relative to a proximal end of the tracking arm. A location of a distal end of an end effector of a procedure tool is determined in the three-dimensional space, wherein the procedure tool is engaged with the distal end of a robot arm, and wherein a proximal end of the robot arm is disposed in a known relation to the proximal end of the tracking arm. A trajectory of the end effector is established relative to the location of the distal end thereof. A plan is formed for a procedure to be performed by the end effector at a location of a physical point at the site or on the object, wherein the procedure includes a route traversed by the end effector to and from the site or the object and during the procedure performed by the end effector at the location and in the established trajectory of the end effector at the physical point at the site or on the object. The procedure is performed at the site or on the object according to the plan, including traversing the route and orienting the end effector in the established trajectory, using the end effector of the procedure tool engaged with the distal end of the robot arm.

The present disclosure thus includes, without limitation, the following embodiments:

Embodiment 1: A method of forming a plan for performing a procedure, the method comprising arranging a distal end of a tracking arm in communication with a reference location at or adjacent to a site or an object received at the site, the reference location being disposed in a relation to a proximal end of the tracking arm in a three-dimensional space; contacting each of a plurality of physical points at or adjacent to the site or the object with a distal end of an end effector of a procedure tool engaged with a distal end of a robot arm, a proximal end of the robot arm being disposed in a known relation to the proximal end of the tracking arm; determining a location of the distal end of the end effector in the three-dimensional space concurrently with contacting each of the physical points with the distal end of the end effector, forming a reference frame in the three-dimensional space, in relation to the reference location, from the locations of the physical points, and to include a location of the site or the object within the reference frame; and forming a plan for a procedure to be performed by the end effector on the site or the object, within the reference frame and relative to the reference location, the procedure including a route traversed by the end effector to and from the site or the object and during the procedure performed on the site or the object by the end effector, and a trajectory of the end effector during the procedure.

Embodiment 2: The method of any preceding embodiment, or any combination of preceding embodiments, wherein contacting each of the physical points with the distal end of the end effector of the procedure tool comprises contacting each of the physical points with the distal end of the end effector, the end effector comprising a drill bit or an abrading bit, of the procedure tool, the procedure tool comprising a drilling device.

Embodiment 3: The method of any preceding embodiment, or any combination of preceding embodiments, wherein the robot arm comprises a plurality of interconnected arm segments having one or more position sensors engaged therewith, and wherein determining the location of the distal end of the end effector comprises determining the location of the distal end of the end effector, relative to the proximal end of the robot arm, from position data from the one or more position sensors.

Embodiment 4: The method of any preceding embodiment, or any combination of preceding embodiments, wherein the tracking arm comprises a plurality of interconnected arm segments having one or more position sensors engaged therewith, and wherein the method comprises determining a location of the distal end of the tracking arm, relative to the proximal end thereof, from position data from the one or more position sensors.

Embodiment 5: The method of any preceding embodiment, or any combination of preceding embodiments, wherein determining the location of the distal end of the end effector in the three-dimensional space comprises determining a location of the site or the object in the three-dimensional space via the end effector, in relation to the reference location, the reference location being physically engaged with the distal end of the tracking arm.

Embodiment 6: The method of any preceding embodiment, or any combination of preceding embodiments, wherein the distal end of the tracking arm is spaced-apart from the site or the object and includes a detector device engaged therewith, the detector device being arranged in communication with the site or the object, and wherein determining the location of the distal end of the end effector comprises determining a location of the site or the object in the three-dimensional space via the end effector, in relation to the reference location, the reference location being detected by the detector device engaged with the distal end of the tracking arm.

Embodiment 7: The method of any preceding embodiment, or any combination of preceding embodiments, wherein determining the location of the distal end of the end effector comprises determining the location of the distal end of the end effector, relative to the reference location determined via the tracking arm, in the three-dimensional space.

Embodiment 8: A method of performing a procedure, the method comprising arranging a distal end of a tracking arm in communication with a reference location at or adjacent to a site or an object received at the site, the reference location being disposed in a three-dimensional space relative to a proximal end of the tracking arm; contacting each of a plurality of physical points at or adjacent to the site or the object with a distal end of an end effector of a procedure tool engaged with a distal end of a robot arm, a proximal end of the robot arm being disposed in a known relation to the proximal end of the tracking arm; determining a location of the distal end of the end effector in the three-dimensional space concurrently with contacting each of the physical points with the distal end of the end effector, forming a reference frame in the three-dimensional space, in relation to the reference location, from the locations of the physical points, and to include a location of the site or the object within the reference frame; forming a plan for a procedure to be performed by the end effector on the site or the object, within the reference frame and relative to the reference location, the procedure including a route traversed by the end effector to and from the site or the object and during the procedure performed on the site or the object by the end effector, and a trajectory of the end effector during the procedure; and performing the procedure on the site or the object according to the plan, including traversing the route, using the end effector of the procedure tool engaged with the distal end of the robot arm.

Embodiment 9: The method of any preceding embodiment, or any combination of preceding embodiments, wherein contacting each of the physical points with the distal end of the end effector of the procedure tool comprises contacting each of the physical points with the distal end of the end effector, the end effector comprising a drill bit or an abrading bit, of the procedure tool, the procedure tool comprising a drilling device.

Embodiment 10: The method of any preceding embodiment, or any combination of preceding embodiments, wherein the robot arm comprises a plurality of interconnected arm segments having one or more position sensors engaged therewith, and wherein determining the location of the distal end of the end effector comprises determining the location of the distal end of the end effector, relative to the proximal end of the robot arm, from position data from the one or more position sensors.

Embodiment 11: The method of any preceding embodiment, or any combination of preceding embodiments, wherein the tracking arm comprises a plurality of interconnected arm segments having one or more position sensors engaged therewith, and wherein the method comprises determining a location of the distal end of the tracking arm, relative to the proximal end thereof, from position data from the one or more position sensors.

Embodiment 12: The method of any preceding embodiment, or any combination of preceding embodiments, wherein determining the location of the distal end of the end effector in the three-dimensional space comprises determining a location of the site or the object in the three-dimensional space via the end effector, in relation to the reference location, the reference location being physically engaged with the distal end of the tracking arm.

Embodiment 13: The method of any preceding embodiment, or any combination of preceding embodiments, wherein the distal end of the tracking arm is spaced-apart from the site or the object and includes a detector device engaged therewith, the detector device being arranged in communication with the site or the object, and wherein determining the location of the distal end of the end effector comprises determining a location of the site or the object in the three-dimensional space via the end effector, in relation to the reference location, the reference location being detected by the detector device engaged with the distal end of the tracking arm.

Embodiment 14: The method of any preceding embodiment, or any combination of preceding embodiments, wherein determining the location of the distal end of the end effector comprises determining the location of the distal end of the end effector, relative to the reference location determined via the tracking arm, in the three-dimensional space.

Embodiment 15: The method of any preceding embodiment, or any combination of preceding embodiments, wherein performing the procedure comprises performing the procedure on the site or the object according to the plan, relative to the reference location determined via the tracking arm, including traversing the route, using the end effector of the procedure tool engaged with the distal end of the robot arm.

Embodiment 16: The method of any preceding embodiment, or any combination of preceding embodiments, comprising adjusting the plan during performance of the procedure in response to movement of the site or the object, as determined via movement of the reference location determined via the tracking arm.

Embodiment 17: The method of any preceding embodiment, or any combination of preceding embodiments, comprising providing feedback in response to the procedure device deviating from the plan during performance of the procedure, including traversing the route.

Embodiment 18: The method of any preceding embodiment, or any combination of preceding embodiments, wherein providing feedback comprises directing haptic feedback or tactile feedback through the procedure device.

Embodiment 19: The method of any preceding embodiment, or any combination of preceding embodiments, wherein providing feedback comprises providing visual feedback or aural feedback through a deviation indication device.

Embodiment 20: The method of any preceding embodiment, or any combination of preceding embodiments, wherein providing feedback comprises restricting movement of the procedure device by providing resistance to motion via the robot arm.

Embodiment 21: A method of performing a procedure, the method comprising arranging a distal end of a tracking arm in communication with a reference location at or adjacent to a site or an object received at the site, the reference location being disposed in a three-dimensional space relative to a proximal end of the tracking arm; contacting a physical point at the site or on the object with a distal end of an end effector of a procedure tool engaged with a distal end of a robot arm, a proximal end of the robot arm being disposed in a known relation to the proximal end of the tracking arm; determining a location of the distal end of the end effector in the three-dimensional space concurrently with contacting the physical point with the distal end of the end effector, establishing a trajectory of the end effector, relative to the physical point at the site or on the object, for performing a procedure at the site or on the object; forming a plan for the procedure to be performed by the end effector at the location of the physical point at the site or on the object, the procedure including a route traversed by the end effector to and from the site or the object and during the procedure performed by the end effector at the location and in the established trajectory at the physical point at the site or on the object; and performing the procedure at the site or on the object according to the plan, including traversing the route, using the end effector of the procedure tool engaged with the distal end of the robot arm.

Embodiment 22: The method of any preceding embodiment, or any combination of preceding embodiments, wherein contacting the physical point with the distal end of the end effector of the procedure tool comprises contacting the physical point with the distal end of the end effector, the end effector comprising a drill bit or an abrading bit, of the procedure tool, the procedure tool comprising a drilling device.

Embodiment 23: The method of any preceding embodiment, or any combination of preceding embodiments, wherein the robot arm comprises a plurality of interconnected arm segments having one or more position sensors engaged therewith, and wherein determining the location of the distal end of the end effector comprises determining the location of the distal end of the end effector, relative to the proximal end of the robot arm, from position data from the one or more position sensors.

Embodiment 24: The method of any preceding embodiment, or any combination of preceding embodiments, wherein the tracking arm comprises a plurality of interconnected arm segments having one or more position sensors engaged therewith, and wherein the method comprises determining a location of the distal end of the tracking arm, relative to the proximal end thereof, from position data from the one or more position sensors.

Embodiment 25: The method of any preceding embodiment, or any combination of preceding embodiments, wherein determining the location of the distal end of the end effector in the three-dimensional space comprises determining a location of the site or the object in the three-dimensional space via the end effector, in relation to the reference location, the reference location being physically engaged with the distal end of the tracking arm.

Embodiment 26: The method of any preceding embodiment, or any combination of preceding embodiments, wherein the distal end of the tracking arm is spaced-apart from the site or the object and includes a detector device engaged therewith, the detector device being arranged in communication with the site or the object, and wherein determining the location of the distal end of the end effector comprises determining a location of the site or the object in the three-dimensional space via the end effector, in relation to the reference location, the reference location being detected by the detector device engaged with the distal end of the tracking arm.

Embodiment 27: The method of any preceding embodiment, or any combination of preceding embodiments, wherein determining the location of the distal end of the end effector comprises determining the location of the distal end of the end effector, relative to the reference location determined via the tracking arm, in the three-dimensional space.

Embodiment 28: The method of any preceding embodiment, or any combination of preceding embodiments, wherein performing the procedure comprises performing the procedure at the site or on the object according to the plan, relative to the reference location determined via the tracking arm, including traversing the route, using the end effector of the procedure tool engaged with the distal end of the robot arm.

Embodiment 29: The method of any preceding embodiment, or any combination of preceding embodiments, comprising adjusting the plan during performance of the procedure in response to movement of the site or the object, as determined via movement of the reference location determined via the tracking arm.

Embodiment 30: The method of any preceding embodiment, or any combination of preceding embodiments, comprising providing feedback in response to the procedure device deviating from the plan during performance of the procedure, including traversing the route.

Embodiment 31: The method of any preceding embodiment, or any combination of preceding embodiments, wherein providing feedback comprises directing haptic feedback or tactile feedback through the procedure device.

Embodiment 32: The method of any preceding embodiment, or any combination of preceding embodiments, wherein providing feedback comprises providing visual feedback or aural feedback through a deviation indication device.

Embodiment 33: The method of any preceding embodiment, or any combination of preceding embodiments, wherein providing feedback comprises restricting movement of the procedure device by providing resistance to motion via the robot arm.

Embodiment 34: The method of any preceding embodiment, or any combination of preceding embodiments, wherein forming the plan comprises forming the plan for the procedure to be performed by the end effector at the location of each of a plurality of physical points at the site or on the object, and wherein performing the procedure comprises performing the procedure at each of the physical points at the site or on the object using the end effector in the established trajectory such that the procedure is performed in parallel between the physical points.

Embodiment 35: A method of performing a procedure, the method comprising arranging a distal end of a tracking arm in communication with a reference location at or adjacent to a site or an object received at the site, the reference location being disposed in a three-dimensional space relative to a proximal end of the tracking arm; determining a location of a distal end of an end effector of a procedure tool in the three-dimensional space, the procedure tool being engaged with the distal end of a robot arm, a proximal end of the robot arm being disposed in a known relation to the proximal end of the tracking arm; establishing a trajectory of the end effector relative to the location of the distal end thereof; forming a plan for a procedure to be performed by the end effector at a location of a physical point at the site or on the object, the procedure including a route traversed by the end effector to and from the site or the object and during the procedure performed by the end effector at the location and in the established trajectory of the end effector at the physical point at the site or on the object; and performing the procedure at the site or on the object according to the plan, including traversing the route and orienting the end effector in the established trajectory, using the end effector of the procedure tool engaged with the distal end of the robot arm.

Embodiment 36: The method of any preceding embodiment, or any combination of preceding embodiments, wherein establishing the trajectory comprises manually orienting the procedure tool such that the end effector is in a three-dimensional angular arrangement relative to the distal end of the end effector, and storing the angular arrangement of the end effector providing the trajectory for use in forming the plan.

Embodiment 37: The method of any preceding embodiment, or any combination of preceding embodiments, wherein establishing the trajectory comprises determining a three-dimensional angular arrangement of the end effector providing the trajectory; determining a spatial offset of a proximal end of the end effector from the distal end thereof providing the angular arrangement, and storing the spatial offset of the proximal end of the end effector providing the trajectory for use in forming the plan.

Embodiment 38: The method of any preceding embodiment, or any combination of preceding embodiments, wherein determining the location of the distal end of the end effector comprises determining the location of the distal end of the end effector, the end effector comprising a drill bit or an abrading bit, of the procedure tool, the procedure tool comprising a drilling device.

Embodiment 39: The method of any preceding embodiment, or any combination of preceding embodiments, wherein determining the location of the distal end of the end effector comprises contacting the physical point with the distal end of the end effector, and concurrently determining a location of the distal end of the end effector in the three-dimensional space.

Embodiment 40: The method of any preceding embodiment, or any combination of preceding embodiments, wherein the robot arm comprises a plurality of interconnected arm segments having one or more position sensors engaged therewith, and wherein determining the location of the distal end of the end effector comprises determining the location of the distal end of the end effector, relative to the proximal end of the robot arm, from position data from the one or more position sensors.

Embodiment 41: The method of any preceding embodiment, or any combination of preceding embodiments, wherein the tracking arm comprises a plurality of interconnected arm segments having one or more position sensors engaged therewith, and wherein the method comprises determining a location of the distal end of the tracking arm, relative to the proximal end thereof, from position data from the one or more position sensors.

Embodiment 42: The method of any preceding embodiment, or any combination of preceding embodiments, wherein determining the location of the distal end of the end effector in the three-dimensional space comprises determining a location of the site or the object in the three-dimensional space via the end effector, in relation to the reference location, the reference location being physically engaged with the distal end of the tracking arm.

Embodiment 43: The method of any preceding embodiment, or any combination of preceding embodiments, wherein the distal end of the tracking arm is spaced-apart from the site or the object and includes a detector device engaged therewith, the detector device being arranged in communication with the site or the object, and wherein determining the location of the distal end of the end effector comprises determining a location of the site or the object in the three-dimensional space via the end effector, in relation to the reference location, the reference location being detected by the detector device engaged with the distal end of the tracking arm.

Embodiment 44: The method of any preceding embodiment, or any combination of preceding embodiments, wherein determining the location of the distal end of the end effector comprises determining the location of the distal end of the end effector, relative to the reference location determined via the tracking arm, in the three-dimensional space.

Embodiment 45: The method of any preceding embodiment, or any combination of preceding embodiments, wherein performing the procedure comprises performing the procedure at the site or on the object according to the plan, relative to the reference location determined via the tracking arm, including traversing the route and orienting the end effector in the established trajectory, using the end effector of the procedure tool engaged with the distal end of the robot arm.

Embodiment 46: The method of any preceding embodiment, or any combination of preceding embodiments, comprising adjusting the plan during performance of the procedure in response to movement of the site or the object, as determined via movement of the reference location determined via the tracking arm.

Embodiment 47: The method of any preceding embodiment, or any combination of preceding embodiments, comprising providing feedback in response to the procedure device deviating from the plan during performance of the procedure, including traversing the route and orienting the end effector in the established trajectory.

Embodiment 48: The method of any preceding embodiment, or any combination of preceding embodiments, wherein providing feedback comprises directing haptic feedback or tactile feedback through the procedure device.

Embodiment 49: The method of any preceding embodiment, or any combination of preceding embodiments, wherein providing feedback comprises providing visual feedback or aural feedback through a deviation indication device.

Embodiment 50: The method of any preceding embodiment, or any combination of preceding embodiments, wherein providing feedback comprises restricting movement of the procedure device by providing resistance to motion via the robot arm.

Embodiment 51: The method of any preceding embodiment, or any combination of preceding embodiments, wherein forming the plan comprises forming the plan for the procedure to be performed by the end effector at the location of each of a plurality of physical points at the site or on the object, and wherein performing the procedure comprises performing the procedure at each of the physical points at the site or on the object using the end effector in the established trajectory such that the procedure is performed in parallel between the physical points.

These and other features, aspects, and advantages of the present disclosure will be apparent from a reading of the following detailed description together with the accompanying drawings, which are briefly described below. The present disclosure includes any combination of two, three, four, or more features or elements set forth in this disclosure, regardless of whether such features or elements are expressly combined or otherwise recited in a specific embodiment description herein. This disclosure is intended to be read holistically such that any separable features or elements of the disclosure, in any of its aspects and embodiments, should be viewed as intended, namely to be combinable, unless the context of the disclosure clearly dictates otherwise.

It will be appreciated that the summary herein is provided merely for purposes of summarizing some example aspects so as to provide a basic understanding of the disclosure. As such, it will be appreciated that the above described example aspects are merely examples and should not be construed to narrow the scope or spirit of the disclosure in any way. It will be appreciated that the scope of the disclosure encompasses many potential aspects, some of which will be further described below, in addition to those herein summarized. Further, other aspects and advantages of such aspects disclosed herein will become apparent from the following detailed description taken in conjunction with the accompanying drawings which illustrate, by way of example, the principles of the described aspects.

### BRIEF DESCRIPTION OF THE SEVERAL VIEWS OF THE DRAWING(S)

Having thus described the disclosure in general terms, reference will now be made to the accompanying drawings, which are not necessarily drawn to scale, and wherein:

FIGS. 1 and 2 schematically illustrate alternate aspects of a robot system implemented by method aspects of the present disclosure;

FIG. 3 schematically illustrates a method of planning a procedure, according to one aspect of the present disclosure, implementing a robot system as shown in FIGS. 1 and 2;

FIG. 4 schematically illustrates the method of FIG. 3 applied in one example to a dental implant in a jawbone;

FIG. 5 schematically illustrates a reference frame formed according to the method of FIG. 3

FIG. 6 schematically illustrates a method of performing a procedure, according to one aspect of the present disclosure, implementing the method of planning a procedure shown in FIG. 3;

FIG. 7 schematically illustrates a method of performing a procedure, according to another aspect of the present disclosure;

FIGS. 8 and 9 schematically illustrates the method of FIG. 7 applied in one example to a dental implant in a jawbone; and

FIG. 10 schematically illustrates a method of performing a procedure, according to yet another aspect of the present disclosure.

### DETAILED DESCRIPTION OF THE DISCLOSURE

The present disclosure now will be described more fully hereinafter with reference to the accompanying drawings, in which some, but not all aspects of the disclosure are shown. Indeed, the disclosure may be embodied in many different forms and should not be construed as limited to the aspects set forth herein; rather, these aspects are provided so that this disclosure will satisfy applicable legal requirements. Like numbers refer to like elements throughout.

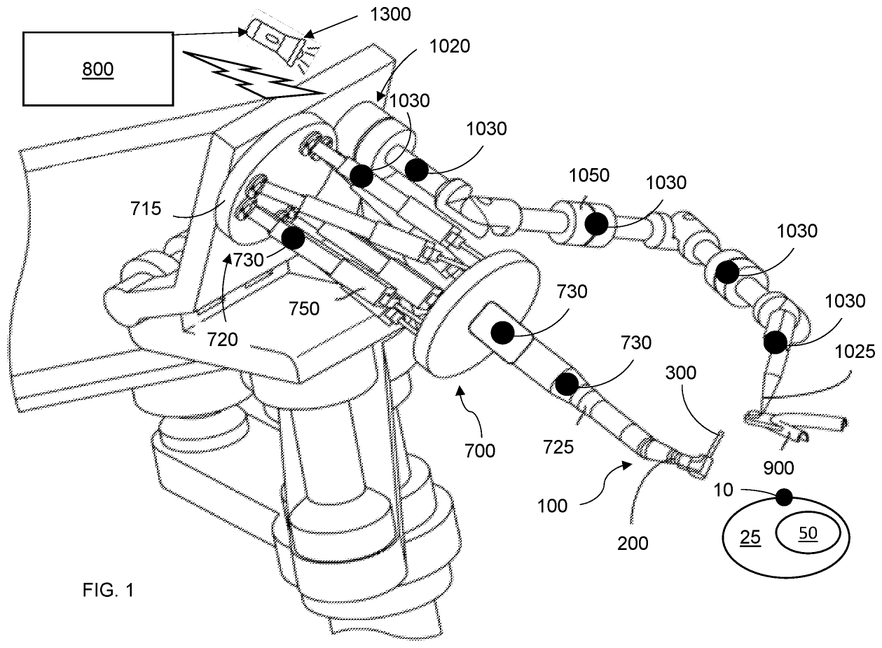

FIG. 1 schematically illustrates a robot system 100 according to one aspect of the present disclosure. Such a system includes a procedure tool 200 having an end effector 300 adapted to interact with a site 25, or an object 50 received at the site 25. The site 25 can be a maxillofacial structure or a dental structure, and the object 50 can be a tooth, a dental implant/crown, or the like.

While aspects of the present disclosure include examples relating the robot system to maxillofacial/dental anatomy or maxillofacial structure, a person of ordinary skill in the art will appreciate that reference to the maxillofacial/dental anatomy or maxillofacial/dental structure, in some aspects, is merely to provide the example of an object interacted with/by the disclosed procedure tool/end effector and/or robot system. Otherwise, reference herein to an “object” is directed and expressly refers to non-human objects. In some examples, such non-human objects are maxillofacial/dental anatomy models or maxillofacial/dental structure models or other non-human representations or reproductions of such anatomy or structure. The disclosed systems and methods herein are implemented to provide, for example, a convenient and effective training tool or training provision for the dental professional to develop their skills in regard to the procedures and tools described herein. Moreover, any methods disclosed and claimed herein are particularly directed to the control and operation of the systems described and claimed herein, wherein such methods are not particularly directed to methods of surgery on humans, but instead to operation of the robot system and/or procedure tool and end effector in relation to the training procedures previously indicated.

Moreover, while aspects of the disclosure illustrate example procedures involving maxillofacial/dental anatomy, one skilled in the art will appreciate that the concept of the robot system and methods disclosed herein may find applicability to other surgical processes not involving dental surgery, such as, for example, orthopedic surgery, ENT surgery, and neurosurgery. As such, the aspects of the disclosure presented herein are merely examples of the applicability of the disclosed concepts and are not intended to be limiting in any manner. That is, aspects of the robot system disclosed herein may be otherwise applicable to various parts of the patient to facilitate other types of surgery, besides dental surgery.

In some aspects, as shown for example in FIGS. 1 and 2, the procedure tool 200 is a drilling device and the end effector 300 is a drill bit or an abrading bit. The procedure tool 200 is engaged with the distal end 725 of an articulating robot arm of the robot system 100, and the end effector 300 of the procedure tool 200 is adapted to interact with the site 25 and/or the object 50 received at the site. A controller 800 is arranged in communication with the articulating robot arm, the procedure tool 200, and a fiducial marker 900 adapted to engage a reference location 10 at or adjacent to the site 25 or the object 50. The controller 800 is arranged, for example, to determine a disposition of the end effector 300 in relation to the fiducial marker 900 during movement of the end effector 300 to interact with the site 25 or the object 50. The controller 800 is further arranged to direct the articulating robot arm to physically control allowable movement of the procedure tool 200, directly relative to the disposition of the end effector 300, with respect to the fiducial marker 900 engaged with the reference location 10, so as to, for instance, account and adapt for movement of the site 25 or the object 50 during the robot procedure. For example, in some aspects, the controller 800 is implemented to develop a plan, procedure, or operation which includes the procedure tool 200/end effector 300 being directed to traverse a route into proximity with the site 25 or the object 50 (and away from the site 25 or the object 50), as well as the subsequent route along which the procedure tool 200/end effector 300 is manipulated into interaction with the site 25 or the object 50, with the end effector 300 in an established trajectory, to perform the plan/procedure/operation.

According to aspects of the present disclosure, the developed plan/procedure/operation details movement, including trajectory, of the procedure tool 200/end effector 300 along a route toward and into engagement with the site 25 or the object 50, while the articulating robot arm (having the procedure tool 200 attached to the distal end 725 thereof) includes structure and functionality to allow the procedure tool 200 to be manually moved along the allowable pathway or route according to the plan/procedure/operation. However, by way of the articulating robot arm, manual movement of the procedure tool 200 outside the allowable pathway or route is restricted, impeded, or otherwise prevented.

In some aspects (see, e.g., FIG. 1), the distal end 1025 of a tracking arm 1050 is physically engaged with the fiducial marker 900. The tracking arm 1050 is a separate and discrete element from the articulating robot arm. Further, the tracking arm 1050, in communication with the controller 800, is thus arranged to cooperate with the controller 800 to determine the spatial relationship between the fiducial marker 900/reference location 10 and the end effector 300 (via the articulating robot arm). In other aspects (see, e.g., FIG. 2), the robot system 100 includes a detector 1000 engaged with a distal end 1025 of a tracking arm 1050, wherein the tracking arm 1050 is a separate and discrete element from the articulating robot arm. The tracking arm 1050 and the detector 1000 are arranged in communication with the controller 800. The detector 1000 is further arranged to cooperate with the tracking arm 1050 to position the detector 1000 in a spaced-apart relationship with the fiducial marker 900 engaged with the reference location 10, to detect the fiducial marker 900 and to cooperate with the controller 800 to determine a spatial relationship between the fiducial marker 900/reference location 10 and the end effector 300 (via the articulating robot arm). The detector 1000, in particular example aspects, is an electrical detector, an electromechanical detector, an electromagnetic detector, an optical detector, an infrared detector, or combinations thereof.

In some aspects, the articulating robot arm has a proximal end 720 and opposed to the distal end 725. One or more sensors 730 is operably engaged with the articulating robot arm and arranged to sense position data associated with the articulating robot arm. For example, the one or more sensors 730 is engaged with one of a plurality of arm members of the articulating robot arm and/or with a joint engaged between arm members, or between arm members and other components of the articulating robot arm (e.g., between the proximal end 720 of the articulating robot arm and a base member 715). In this manner, the position data sensed by the one or more sensors 730 includes, for example, the spatial relationship (e.g., orientation, position, etc.) of the articulating robot arm and/or the components thereof in a three dimensional space. In some instances, the spatial relationship is determined relative to the base member 715 to which the proximal end 720 of the articulating robot arm is mounted. As such, in some aspects, the one or more sensors 730 is engaged with the articulating robot arm such that the position data sensed by the one or more sensors 730 at least indicates the spatial position of at least the distal end 725 of the articulating robot arm in a three dimensional space, and in some instances relative to the base member 715/proximal end 720 of the articulating robot arm. The position of the procedure tool 200/end effector 300, in the three-dimensional space, are related to, known or determined from, or otherwise associated with the position of the distal end 725 of the articulating robot arm determined from the position data of the one or more sensors 730, and the engagement of the procedure tool 200 with the distal end 725 of the articulating robot arm. As such, the location of the distal end of the end effector 300, relative to the proximal end 720 of the robot arm 750, is determined from position data from the one or more position sensors 730.

In some aspects, the tracking arm 1050 has a proximal end 1020 and opposed to the distal end 1025. One or more sensors 1030 is operably engaged with the tracking arm 1050 and arranged to sense position data associated with the tracking arm 1050. For example, the one or more sensors 1030 is engaged with one of a plurality of arm members of the tracking arm 1050 and/or with a joint engaged between arm members, or between arm members and other components of the tracking arm 1050 (e.g., between the proximal end 1020 of the tracking arm 1050 and the base member 715). In this manner, the position data sensed by the one or more sensors 1030 includes, for example, the spatial relationship (e.g., orientation, position, etc.) of the tracking arm 1050 and/or the components thereof in a three dimensional space. In some instances, the spatial relationship is determined relative to the base member 715 to which the proximal end 1020 of the tracking arm 1050 is mounted. As such, in some aspects, the one or more sensors 1030 is engaged with the tracking arm 1050 such that the position data sensed by the one or more sensors 1030 at least indicates the spatial position of at least the distal end 1025 of the tracking arm 750 in a three dimensional space, and in some instances relative to the base member 715/proximal end 1020 of the tracking arm 1050. The position of the reference location 10, in the three-dimensional space, is related to, known or determined from, or otherwise associated with the position of the distal end 1025 of the tracking arm 1050 determined from the position data of the one or more sensors 1030, and the physical engagement between the fiducial marker 900/reference location 10 and the distal end 1025 of the tracking arm 1050, or the detection of the fiducial marker 900/reference location 10 by the detector 1000 engaged with the distal end 1025 of the tracking arm 1050. That is, the location of the distal end 1025 of the tracking arm 1050, relative to the proximal end 1020 thereof, is determined from position data from the one or more position sensors 1030.

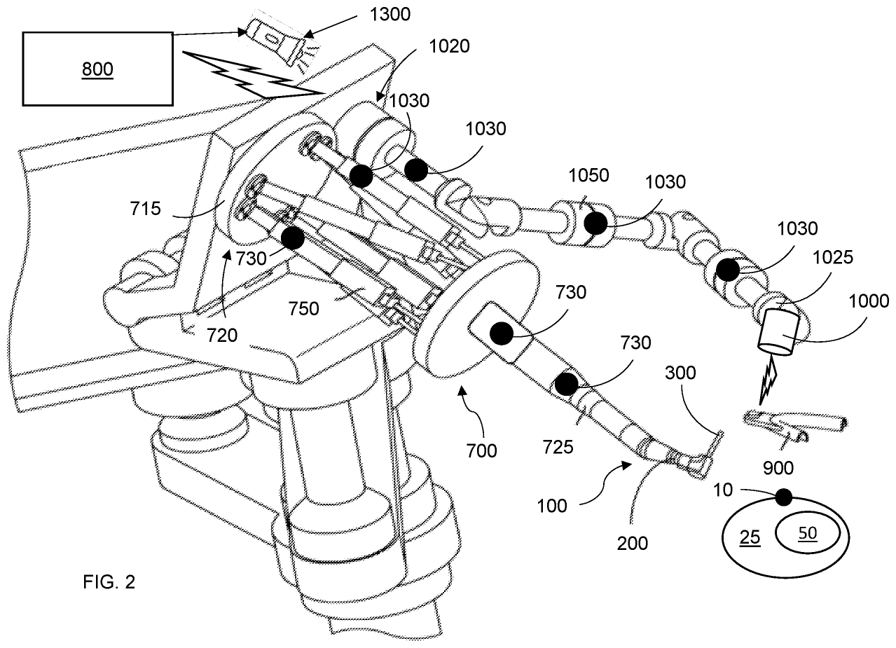

Implementing aspects of the robot system 100 as disclosed herein, one aspect of the present disclosure involves a method of forming a plan for performing a procedure, particularly a method of forming such a plan, without requiring imaging of the site 25/object 50, as shown in FIG. 3. Such a method, in one instance, comprises arranging a distal end 1025 of a tracking arm 1050 in communication with a reference location 10 at or adjacent to a site 25 or an object 50 received at the site 25, wherein the reference location 10 is disposed in a relation to a proximal end 1020 of the tracking arm 1050 in a three-dimensional space (Block 3-100) (i.e., the reference location 10 is disposed in a three-dimensional space relative to the proximal end 1020 of the tracking arm 1050). The location in the three-dimensional space of the distal end 1025 of the end effector 300, relative to the reference location 10, is known or determined via the tracking arm 1050, by way of a physical connection or communication with a fiducial marker 900 engaged with the reference location 10.

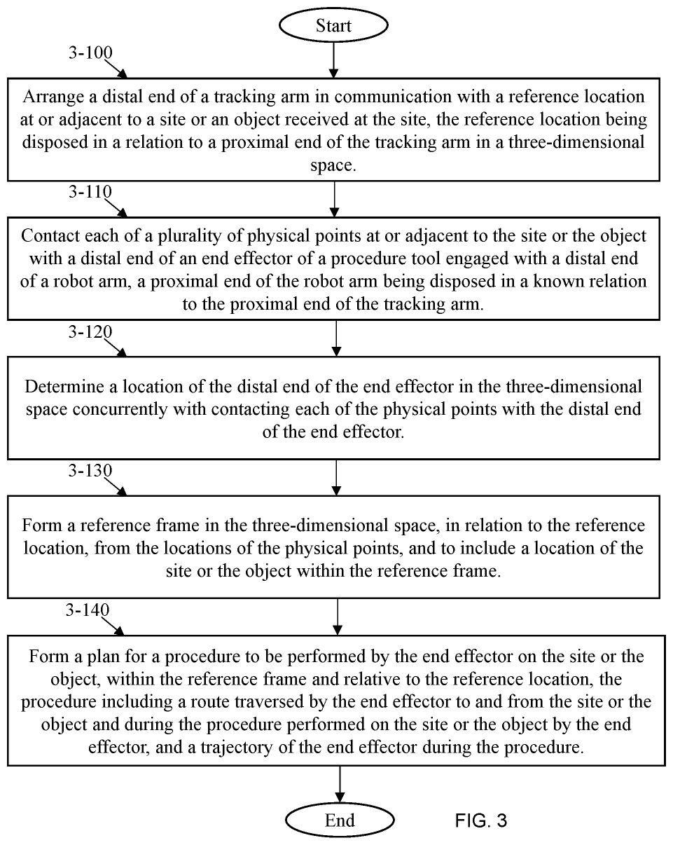

Each of a plurality of physical points 5 at or adjacent to the site 25 or the object 50 is contacted with a distal end of an end effector 300 of a procedure tool 200 engaged with a distal end 725 of a robot arm 750, wherein a proximal end 720 of the robot arm 750 is disposed in a known relation to the proximal end 1020 of the tracking arm 1050 (Block 3-110). A location of the distal end of the end effector 300 in the three-dimensional space is determined concurrently with contacting each of the physical points 5 with the distal end of the end effector 300 (Block 3-120 and FIG. 4). In addition, the site 25 or the object 50 is one of the physical points, and the location of the site 25 or the object 50 is also determined in the three-dimensional space via the end effector 300, in relation to the reference location 10, since the reference location 10 is physically engaged with the distal end 1025 of the tracking arm 1050 (via the fiducial marker 900) or is otherwise detected (via the fiducial marker 900) by the detector device 1000 engaged with the distal end 1025 of the tracking arm 1050.

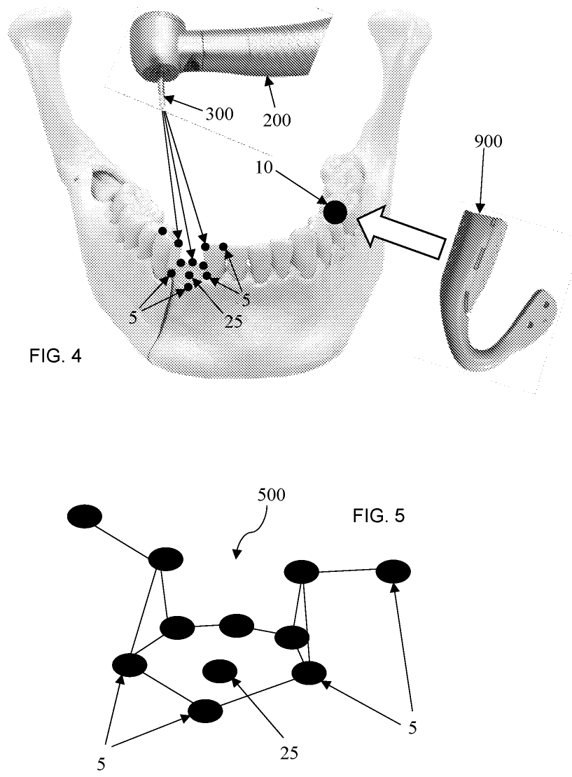

A reference frame 500 (see, e.g., FIG. 5) is then formed in the three-dimensional space, in relation to the reference location 10, from the locations of the physical points 5, wherein a location of the site 25 or the object 50 is also included within the reference frame 500 (Block 3-130). That is, in some instances, the physical points 5 contacted with the distal end of the end effector 300 are selected to surround and/or include the site 25 or the object 50 such that, when formed, the site 25 or the object 50 are disposed within or in relation to the reference frame 500. Once the reference frame 500 is established, a plan is formed for a procedure to be performed by the end effector 300 on the site 25 or the object 50, within the reference frame 500 and relative to the reference location 10, wherein the procedure includes a route traversed by the end effector 300 to and from the site 25 or the object 50 and during the procedure performed on the site 25 or the object 50 by the end effector 300, as well as a trajectory of the end effector 300 during the procedure (Block 3-140). Since both the reference frame 500 and the route(s) of the procedure tool 200/end effector 300 are established with respect to the reference location 10, and since the reference location 10 in the three-dimensional space is monitored during execution of the plan/procedure/operation, the reference frame 500 and the route(s) can be modified or adjusted in real time to account for any movement of the site 25/object 50 as the plan/procedure/operation is being executed.

Accordingly, in such aspects, the plan/procedure/operation is formed, without requiring a separate imaging procedure of the site 25/object 50 as the basis for forming the plan/procedure/operation. That is, the arrangement of the robot system 100, in which the location of the distal end of the end effector 300 is known in relation to the fiducial marker 900 engaged with the site 25/object 50, allows the reference frame 500, established via capture of the location(s) of the distal end of the end effector 300 with each of the physical points 5, to serve as a proxy or replacement for the imaging step. Since the physical points 5 are selected on or about the site 25/object 50, and can include the site 25/object 50 itself, the reference frame 500 is formed as a three-dimensional representation or model of the site 25/object 50. Moreover, since the site 25/object 50 itself can be included as one of the physical points 5, this method of forming the reference frame 500 to include the site 25/object 50 itself provides an accurate three-dimensional representation or model on which to form the plan/procedure/operation. As shown, for example, in FIGS. 4 and 5, the robot system 100 and method can be applied in some instances to placing a dental implant in a model jaw structure, which requires an implant/post to be installed in a jaw (e.g., a site 25) prior to the post receiving a crown thereon. By contacting the physical points 5 on anatomical structures at, near, or adjacent to the site 25 (e.g., the surface of nearby teeth and on the jawbone surface (lingual and buccal)) with the end of a drill bit (end effector 300) of a drill device (procedure tool 200) attached to the distal end 725 of the robot arm 750, locations of those physical points 5 in a three-dimensional space are recorded via the controller 800. The controller 800 can then form a localized reference frame 500 that include the site 25 therein and, based on this reference frame 500 forms a plan directed to, e.g., centering the implant/post being centered at the site 25 in the jawbone, with the implant/post extending out of the jawbone at an angle or trajectory centered between the teeth on either side (mesial and distal). This precise placing of the implant/post thus facilitates the process of placing the crown on the implant/post (i.e., restoring the tooth) by optimizing the alignment between the implant/post and crown in view of the surrounding structures.

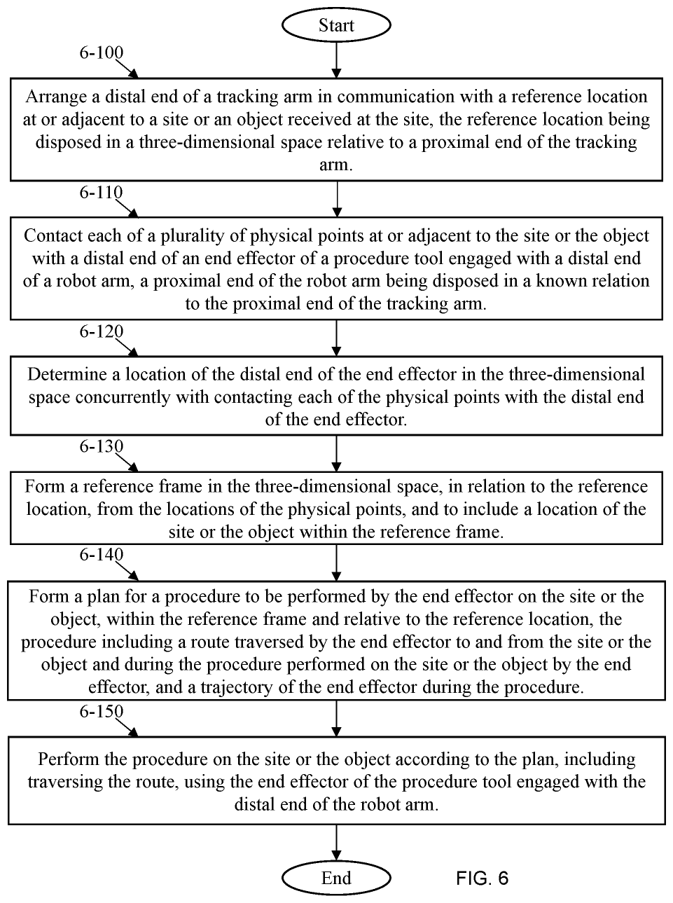

In another aspect, the method of forming the plan by way of establishing a reference frame 500 can be extended to a method of performing a procedure based on that plan. As shown, for example, in FIG. 6, such a method comprises arranging a distal end 1025 of a tracking arm 1050 in communication with a reference location 10 at or adjacent to a site 25 or an object 50 received at the site 25, wherein the reference location 10 is disposed in a three-dimensional space relative to a proximal end 1020 of the tracking arm 1050 (Block 6-100). Each of a plurality of physical points 5 at or adjacent to the site 25 or the object 50 is contacted with a distal end of an end effector 300 of a procedure tool 200 engaged with a distal end 725 of a robot arm 750, wherein a proximal end 720 of the robot arm 750 is disposed in a known relation to the proximal end 1020 of the tracking arm 1050 (Block 6-110). A location of the distal end of the end effector 300 in the three-dimensional space is determined concurrently with contacting each of the physical points 5 with the distal end of the end effector 300. (Block 6-120). A reference frame 500 in the three-dimensional space is then formed in relation to the reference location 10, from the locations of the physical points 5, and to include a location of the site 25 or the object 50 within the reference frame 10 (Block 6-130).

A plan is then formed for a procedure to be performed by the end effector 300 on the site 25 or the object 50, within the reference frame 500 and relative to the reference location 10. The procedure includes a route traversed by the end effector 300 to and from the site 25 or the object 50 and during the procedure performed on the site 25 or the object 50 by the end effector 300, as well as a trajectory or orientation of the end effector 300 during the procedure (Block 6-140). The procedure is then performed on the site 25 or the object 50 according to the plan, including traversing the route, using the end effector 300 of the procedure tool 200 engaged with the distal end 725 of the robot arm 750 (Block 6-150). That is, the procedure is performed on the site 25 or the object 50 according to the plan, relative to the reference location 10 determined via the tracking arm 1050, including traversing the route(s), using the end effector 300 of the procedure tool 200 engaged with the distal end 725 of the robot arm 750.

Since the communication with the fiducial marker 900/reference location 10 remains during performance of the procedure, the plan, including the route(s) traversed by the procedure tool 200/end effector 300 can be adjusted during performance of the procedure in response to movement of the site 25 or the object 50, as determined via movement of the reference location 10 determined via the tracking arm 1050. Moreover, since the procedure tool 200/robot arm 750 can be manually manipulated to conduct the procedure, the robot system 100 can be arranged to provide feedback in response to the procedure device 200 deviating from the plan (e.g., deviating from the route(s)) during performance of the procedure, including traversing the route(s). For example, haptic feedback or tactile feedback can be directed through the procedure device 200 by the controller 800, visual feedback or aural feedback through a deviation indication device (see, e.g., element 1300 in FIGS. 1 and 2) by the controller 800, and/or movement of the procedure device 200 can be restricted by providing resistance to motion via the robot arm 750.

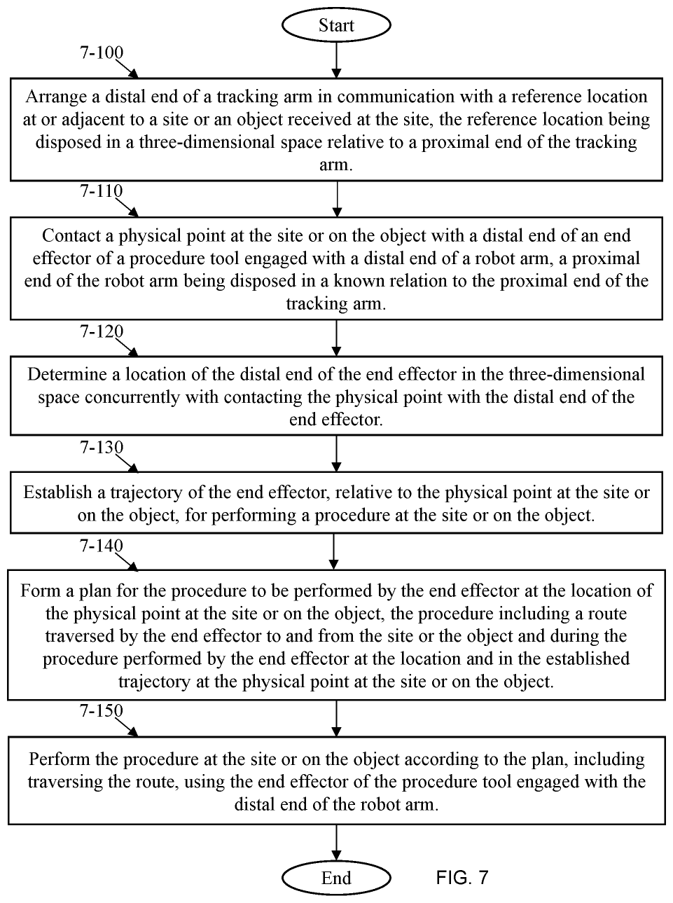

In still another aspect of the present disclosure, a method of performing a procedure can be implemented without the formation of a reference frame 500. For example, as shown in FIGS. 7-9, such as method comprises arranging a distal end 1025 of a tracking arm 1050 in communication with a reference location 10 at or adjacent to a site 25 or an object 50 received at the site 25, wherein the reference location 10 is disposed in a three-dimensional space relative to a proximal end 1020 of the tracking arm 1050 (Block 7-100). A physical point at the site 25 or on the object 50 (e.g., the site 25 itself) is contacted with a distal end of an end effector 300 of a procedure tool 200 engaged with a distal end 725 of a robot arm 750, wherein a proximal end 720 of the robot arm 750 is disposed in a known relation to the proximal end 1020 of the tracking arm 1050 (Block 7-110). A location of the distal end of the end effector 300 in the three-dimensional space is determined concurrently with contacting the physical point (e.g., the site 25) with the distal end of the end effector 300 (Block 7-120).

In conjunction with contacting the physical point (e.g., the site 25) with the distal end of the end effector 300, a trajectory or orientation (see, e.g., element 1500 in FIG. 9) of the end effector 300, relative to the physical point at the site 25 or on the object 50, is established for performing a procedure at the site 25 or on the object 50 (e.g., a drill bit can be placed in contact with the site 25, and then the drilling device/drill bit manually aligned such that the drill bit is aligned in a desired orientation/direction in the three-dimensional space to drill a hole at the site 25) (Block 7-130). A plan is then formed for the procedure to be performed by the end effector 300 at the location of the physical point at the site 25 or on the object 50 (e.g., by providing a signal via an actuator—see, e.g., element 1400 in FIG. 9) to the controller 800 when the end effector 300 is in contact with the site 25 and oriented at the desired angle (e.g., trajectory or orientation 1500 in the three dimensional space) with respect to the site 25) (Block 7-140). The procedure can also include a route traversed by the end effector 300 to and from the site 25 or the object 50 and during the procedure performed by the end effector 300 at the location and in the established trajectory 1500 at the physical point at the site 25 or on the object 50. The procedure is then performed at the site 25 or on the object 50 according to the plan, including traversing the route(s), using the end effector 300 of the procedure tool 200 engaged with the distal end 725 of the robot arm 750 (Block 7-150). That is, the procedure is performed on the site 25 or the object 50 according to the plan, relative to the reference location 10 determined via the tracking arm 1050, including traversing the route(s) with the end effector 300 in the established trajectory 1500, using the end effector 300 of the procedure tool 200 engaged with the distal end 725 of the robot arm 750.

Since the communication with the fiducial marker 900/reference location 10 remains during performance of the procedure, the plan, including the route(s) traversed by the procedure tool 200/end effector 300 and the trajectory 1500 of the end effector 300, can be adjusted during performance of the procedure in response to movement of the site 25 or the object 50, as determined via movement of the reference location 10 determined via the tracking arm 1050. Moreover, since the procedure tool 200/robot arm 750 can be manually manipulated to conduct the procedure, the robot system 100 can be arranged to provide feedback in response to the procedure device 200 deviating from the plan (e.g., deviating from the route(s)) during performance of the procedure, including traversing the route(s). For example, haptic feedback or tactile feedback can be directed through the procedure device 200 by the controller 800, visual feedback or aural feedback through a deviation indication device (see, e.g., element 1300 in FIGS. 1 and 2) by the controller 800, and/or movement of the procedure device 200 can be restricted by providing resistance to motion via the robot arm 750

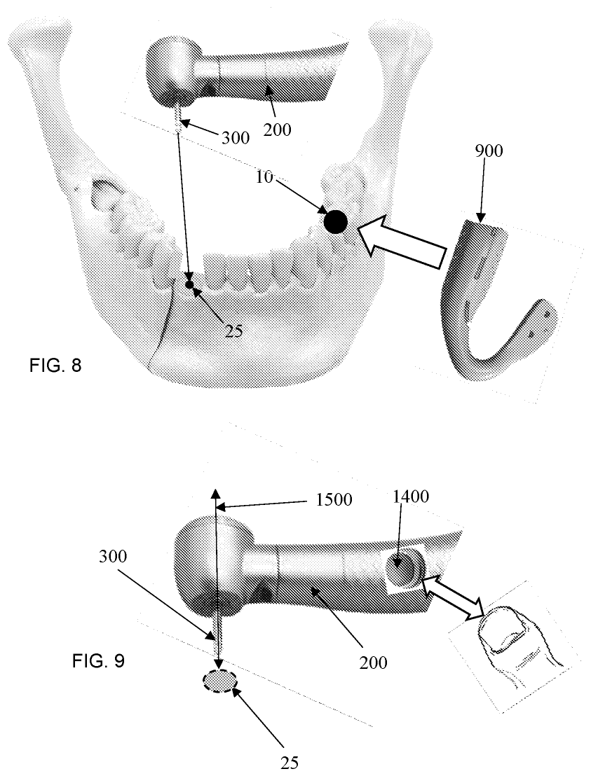

Accordingly, in such aspects, as shown, for example, in FIGS. 8 and 9, the robot system 100 and method can be applied in some instances to placing a dental implant in a model jaw structure, which requires an implant/post to be installed in a jaw (e.g., a site 25) prior to the post receiving a crown thereon. By contacting the site 25 (e.g., on the jawbone surface) with the end of a drill bit (end effector 300) of a drill device (procedure tool 200) attached to the distal end 725 of the robot arm 750, and establishing the desired trajectory/orientation of the drill bit, the location of the implant site 25 in a three-dimensional space can be recorded via the controller 800 in response to the actuator 1400. If desired, the controller 800 can then form a plan directed to, e.g., placing the implant/post at the site 25 in the jawbone. However, in an alternate aspect, the imaging process, as well as the planning process previously disclosed herein can both be bypassed, and the procedure/operation conducted immediately upon establishing the location of the site 25 and the trajectory/orientation of the end effector 300. In some instances, the distance of movement of the end effector 300 along the orientation/trajectory can also be established, e.g., by input to the controller 800. In this manner, the robot system 100 can return the end effector 300 to the same position/orientation/trajectory, for example, if progressively larger holes need to be drilled at the site 25.

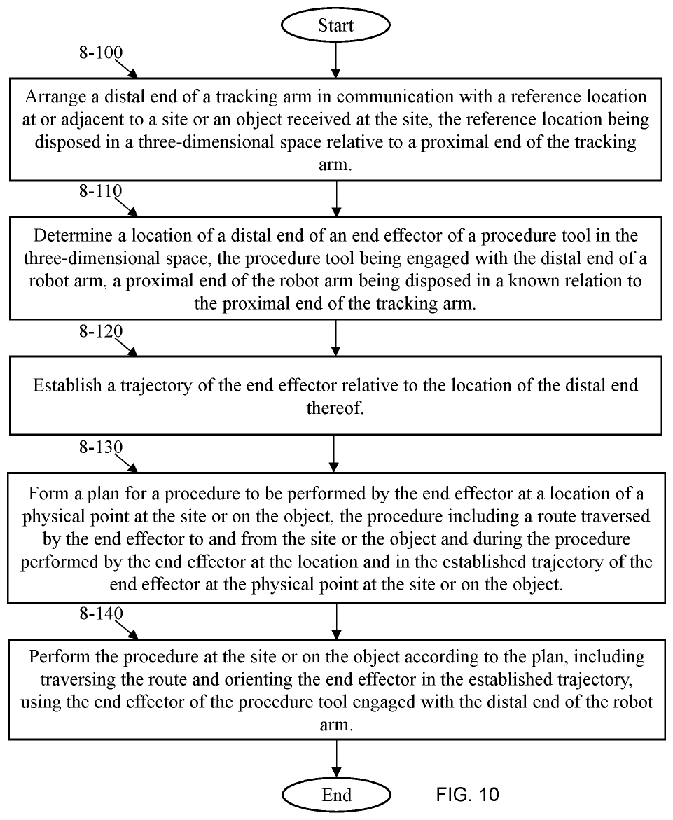

In yet another aspect of the present disclosure, a method of performing a procedure can be implemented without contacting a physical point at the site 25 or on the object 50 (e.g., the site 25 itself) with the distal end of the end effector 300, in order to determine the location of the distal end of the end effector 300 in the three-dimensional space. For example, as shown in FIG. 10, such as method comprises arranging a distal end 1025 of a tracking arm 1050 in communication with a reference location 10 at or adjacent to a site 25 or an object 50 received at the site 25, wherein the reference location 10 is disposed in a three-dimensional space relative to a proximal end 1020 of the tracking arm 1050 (Block 8-100). A location of a distal end of an end effector 300 of a procedure tool 200 is determined in the three-dimensional space, wherein the procedure tool 200 is engaged with the distal end 725 of a robot arm 750, and wherein a proximal end 720 of the robot arm 750 is disposed in a known relation to the proximal end 1020 of the tracking arm 1050 (Block 8-110).

That is, since the robot arm 750 comprises a plurality of interconnected arm segments having one or more position sensors 730 engaged therewith (wherein the location of the distal end of the end effector 300 relative to the proximal end 720 of the robot arm 750 can be determined from position data from the one or more position sensors 730), and since the tracking arm 1050 comprises a plurality of interconnected arm segments having one or more position sensors 1030 engaged therewith (wherein the location of the distal end 1025 of the tracking arm 1050, relative to the proximal end 1020 thereof, can be determined from position data from the one or more position sensors 1030), the location of the distal end of the end effector 300 in the three-dimensional space in relation to the reference location 10 can be determined at any point in the three-dimensional space. For example, the location of the distal end of the end effector 300 in the three-dimensional space can be continually monitored by way of the robot arm 750, or the location of the distal end of the end effector 300 in the three-dimensional space can be determined on-demand upon actuation of the actuator 1400 (see, e.g., FIG. 9). In such instances, the location of the distal end of the end effector 300 is determined in free space (e.g., at any location within the three-dimensional space). However, in other aspects, the location of the distal end of the end effector 300 can be determined by contacting a physical point (e.g., the site 25) with the distal end of the end effector 300, and concurrently determining a location of the distal end of the end effector 300 in the three-dimensional space.

Upon the location of the distal end of the end effector 300 being determined, a trajectory (see, e.g., element 1500 in FIG. 9) of the end effector 300 is then established relative to the location of the distal end thereof (Block 8-120). That is, in some aspects, the trajectory 1500 is established by manually orienting the procedure tool 200 such that the end effector 300 is in a three-dimensional angular arrangement relative to the distal end of the end effector 300, wherein the angular arrangement of the end effector 300 providing the trajectory 1500 is stored (e.g., upon actuation of the actuator 1400) for use in forming a plan for the procedure. In other aspects, the trajectory 1500 is established by determining a three-dimensional angular arrangement of the end effector 300 providing the desired trajectory, and then determining a spatial offset of a proximal end of the end effector 300, from the distal end thereof, for providing the angular arrangement. The spatial offset of the proximal end of the end effector 300 providing the trajectory 1500 is then stored for use in forming a plan for the procedure.

A plan is then formed for the procedure to be performed by the end effector 300 at the location of the physical point at the site 25 or on the object 50 (e.g., by providing a signal via actuator (see, e.g., element 1400 in FIG. 9) to the controller 800 when the end effector 300 is oriented at the desired angle with respect to the site 25) (Block 8-130). The procedure can also include a route traversed by the end effector 300 to and from the site 25 or the object 50 and during the procedure performed by the end effector 300 at the location and in the established trajectory of the end effector 300 at the physical point at the site 25 or on the object 50. The procedure is then performed at the site 25 or on the object 50 according to the plan, including traversing the route(s) and orienting the end effector 300 in the established trajectory 1500, using the end effector 300 of the procedure tool 200 engaged with the distal end 725 of the robot arm 750 (Block 8-140). That is, the procedure is performed on the site 25 or the object 50 according to the plan, relative to the reference location 10 determined via the tracking arm 1050, including traversing the route(s) and orienting the end effector 300 in the established trajectory 1500, using the end effector 300 of the procedure tool 200 engaged with the distal end 725 of the robot arm 750.

Since the communication with the fiducial marker 900/reference location 10 remains during performance of the procedure, the plan, including the route(s) traversed by the procedure tool 200/end effector 300 and the trajectory of the end effector 300 can be adjusted during performance of the procedure in response to movement of the site 25 or the object 50, as determined via movement of the reference location 10 determined via the tracking arm 1050. Moreover, since the procedure tool 200/robot arm 750 can be manually manipulated to conduct the procedure, the robot system 100 can be arranged to provide feedback in response to the procedure device 200 deviating from the plan (e.g., deviating from the route(s) and/or the trajectory of the end effector 300) during performance of the procedure, including traversing the route(s) and orienting the end effector 300 in the established trajectory 1500. For example, haptic feedback or tactile feedback can be directed through the procedure device 200 by the controller 800, visual feedback or aural feedback through a deviation indication device (see, e.g., element 1300 in FIGS. 1 and 2) by the controller 800, and/or movement of the procedure device 200 can be restricted by providing resistance to motion via the robot arm 750.

Since the method illustrated in FIG. 10 can be accomplished without contacting a physical point at the site 25 or on the object 50 (e.g., the site 25 itself) with the distal end of the end effector 300, in order to determine the location of the distal end of the end effector 300 in the three-dimensional space (e.g., for establishing the trajectory of the end effector 300), such a method can also provide an opportunity for conducting the procedure at multiple sites 25 with the end effector 300 in the same trajectory 1500. That is, in some instances, a prosthetic restoration (e.g., a denture) is fitted to multiple implants/anchors, wherein fitting the prosthetic restoration is facilitated if the involved anchors/implants are arranged substantially parallel to each other. The establishment of the trajectory 1500 of the end effector 300 independently of contact with a physical point to determine the location of the distal end of the end effector 300 thus allows the procedure to be conducted at multiple sites 25 with the end effector 300 oriented in the same trajectory 1500 (e.g., multiple bores can be drilled using the drill bit oriented in the same trajectory such that the bores are parallel to each other in the three-dimensional space, where the implants/anchors received by those bores will be substantially parallel to each other for facilitating receipt of the prosthetic restoration). More particularly, in some aspects, the plan can be formed for the procedure to be performed by the end effector 300 at the location of each of a plurality of physical points (e.g., multiple sites 25) at the site 25 or on the object 50. As such, the procedure can be performed at each of the physical points at the site 25 or on the object 50, using the end effector 300 in the same established trajectory, such that the procedure is performed in parallel between the physical points (e.g., parallel bores are formed).

Aspects of the present disclosure thus provide, for example, methods of planning and executing a procedure (e.g., a dental or maxillofacial procedure), and particularly a robot-assisted or robot-implemented procedure, that can be accomplished, without requiring one or more forms of imaging of the site (e.g., jaws or maxillofacial bone structure). In some aspects, the stage of planning the procedure can also be bypassed such that the procedure can be executed immediately in a repeatable manner. In some instances, since such methods can be accomplished while the site is established in a reference frame known to the robot system, the planning and conducting of the procedure on the site can be conducted in a compact time frame and without requiring the site to be subject to a separate imaging system beforehand, at added time and expense. Moreover, such methods are ergonomically friendly, expedient, and convenient for the dental professional, for example, by minimizing the steps necessary to effectuate the plan and obviating the need for interchanging instruments between planning and conducting the procedure.

Many modifications and other embodiments of the inventions set forth herein will come to mind to one skilled in the art to which these disclosed embodiments pertain having the benefit of the teachings presented in the foregoing descriptions and the associated drawings. Therefore, it is to be understood that embodiments of the invention are not to be limited to the specific embodiments disclosed and that modifications and other embodiments are intended to be included within the scope of the invention. Moreover, although the foregoing descriptions and the associated drawings describe example embodiments in the context of certain example combinations of elements and/or functions, it should be appreciated that different combinations of elements and/or functions may be provided by alternative embodiments without departing from the scope of the disclosure. In this regard, for example, different combinations of elements and/or functions than those explicitly described above are also contemplated within the scope of the disclosure. Although specific terms are employed herein, they are used in a generic and descriptive sense only and not for purposes of limitation.

It should be understood that although the terms first, second, etc. may be used herein to describe various steps or calculations, these steps or calculations should not be limited by these terms. These terms are only used to distinguish one operation or calculation from another. For example, a first calculation may be termed a second calculation, and, similarly, a second step may be termed a first step, without departing from the scope of this disclosure. As used herein, the term “and/or” and the “/” symbol includes any and all combinations of one or more of the associated listed items.

As used herein, the singular forms “a”, “an” and “the” are intended to include the plural forms as well, unless the context clearly indicates otherwise. It will be further understood that the terms “comprises”, “comprising”, “includes”, and/or “including”, when used herein, specify the presence of stated features, integers, steps, operations, elements, and/or components, but do not preclude the presence or addition of one or more other features, integers, steps, operations, elements, components, and/or groups thereof. Therefore, the terminology used herein is for the purpose of describing particular embodiments only and is not intended to be limiting.

That which is claimed:

1. A method of forming a plan for performing a procedure, the method comprising:
arranging a distal end of a tracking arm in communication with a reference location at or adjacent to a site or an object received at the site, the reference location being disposed in a relation to a proximal end of the tracking arm in a three-dimensional space;
contacting each of a plurality of physical points at or adjacent to the site or the object with a distal end of an end effector of a procedure tool engaged with a distal end of a robot arm, a proximal end of the robot arm being disposed in a known relation to the proximal end of the tracking arm;
determining a location of the distal end of the end effector in the three-dimensional space concurrently with contacting each of the physical points with the distal end of the end effector,
forming a reference frame in the three-dimensional space, in relation to the reference location, from the locations of the physical points, and to include a location of the site or the object within the reference frame; and
forming a plan for a procedure to be performed by the end effector on the site or the object, within the reference frame and relative to the reference location, the procedure including a route traversed by the end effector to and from the site or the object and during the procedure performed on the site or the object by the end effector, and a trajectory of the end effector during the procedure.

2. The method of claim 1, wherein contacting each of the physical points with the distal end of the end effector of the procedure tool comprises contacting each of the physical points with the distal end of the end effector, the end effector comprising a drill bit or an abrading bit, of the procedure tool, the procedure tool comprising a drilling device.

3. The method of claim 1, wherein the robot arm comprises a plurality of interconnected arm segments having one or more position sensors engaged therewith, and wherein determining the location of the distal end of the end effector comprises determining the location of the distal end of the end effector, relative to the proximal end of the robot arm, from position data from the one or more position sensors.

4. The method of claim 1, wherein the tracking arm comprises a plurality of interconnected arm segments having one or more position sensors engaged therewith, and wherein the method comprises determining a location of the distal end of the tracking arm, relative to the proximal end thereof, from position data from the one or more position sensors.

5. The method of claim 1, wherein determining the location of the distal end of the end effector in the three-dimensional space comprises determining a location of the site or the object in the three-dimensional space via the end effector, in relation to the reference location, the reference location being physically engaged with the distal end of the tracking arm.

6. The method of claim 1, wherein the distal end of the tracking arm is spaced-apart from the site or the object and includes a detector device engaged therewith, the detector device being arranged in communication with the site or the object, and wherein determining the location of the distal end of the end effector comprises determining a location of the site or the object in the three-dimensional space via the end effector, in relation to the reference location, the reference location being detected by the detector device engaged with the distal end of the tracking arm.

7. The method of claim 1, wherein determining the location of the distal end of the end effector comprises determining the location of the distal end of the end effector, relative to the reference location determined via the tracking arm, in the three-dimensional space.

8. A method of performing a procedure, the method comprising:
arranging a distal end of a tracking arm in communication with a reference location at or adjacent to a site or an object received at the site, the reference location being disposed in a three-dimensional space relative to a proximal end of the tracking arm;
contacting each of a plurality of physical points at or adjacent to the site or the object with a distal end of an end effector of a procedure tool engaged with a distal end of a robot arm, a proximal end of the robot arm being disposed in a known relation to the proximal end of the tracking arm;
determining a location of the distal end of the end effector in the three-dimensional space concurrently with contacting each of the physical points with the distal end of the end effector,
forming a reference frame in the three-dimensional space, in relation to the reference location, from the locations of the physical points, and to include a location of the site or the object within the reference frame;
forming a plan for a procedure to be performed by the end effector on the site or the object, within the reference frame and relative to the reference location, the procedure including a route traversed by the end effector to and from the site or the object and during the procedure performed on the site or the object by the end effector, and a trajectory of the end effector during the procedure; and
performing the procedure on the site or the object according to the plan, including traversing the route, using the end effector of the procedure tool engaged with the distal end of the robot arm.

9. The method of claim 8, wherein contacting each of the physical points with the distal end of the end effector of the procedure tool comprises contacting each of the physical points with the distal end of the end effector, the end effector comprising a drill bit or an abrading bit, of the procedure tool, the procedure tool comprising a drilling device.

10. The method of claim 8, wherein the robot arm comprises a plurality of interconnected arm segments having one or more position sensors engaged therewith, and wherein determining the location of the distal end of the end effector comprises determining the location of the distal end of the end effector, relative to the proximal end of the robot arm, from position data from the one or more position sensors.

11. The method of claim 8, wherein the tracking arm comprises a plurality of interconnected arm segments having one or more position sensors engaged therewith, and wherein the method comprises determining a location of the distal end of the tracking arm, relative to the proximal end thereof, from position data from the one or more position sensors.

12. The method of claim 8, wherein determining the location of the distal end of the end effector in the three-dimensional space comprises determining a location of the site or the object in the three-dimensional space via the end effector, in relation to the reference location, the reference location being physically engaged with the distal end of the tracking arm.

13. The method of claim 8, wherein the distal end of the tracking arm is spaced-apart from the site or the object and includes a detector device engaged therewith, the detector device being arranged in communication with the site or the object, and wherein determining the location of the distal end of the end effector comprises determining a location of the site or the object in the three-dimensional space via the end effector, in relation to the reference location, the reference location being detected by the detector device engaged with the distal end of the tracking arm.

14. The method of claim 8, wherein determining the location of the distal end of the end effector comprises determining the location of the distal end of the end effector, relative to the reference location determined via the tracking arm, in the three-dimensional space.

15. The method of claim 8, wherein performing the procedure comprises performing the procedure on the site or the object according to the plan, relative to the reference location determined via the tracking arm, including traversing the route, using the end effector of the procedure tool engaged with the distal end of the robot arm.

16. The method of claim 15, comprising adjusting the plan during performance of the procedure in response to movement of the site or the object, as determined via movement of the reference location determined via the tracking arm.

17. The method of claim 8, comprising providing feedback in response to the procedure device deviating from the plan during performance of the procedure, including traversing the route.

18. The method of claim 17, wherein providing feedback comprises directing haptic feedback or tactile feedback through the procedure device.

19. The method of claim 17, wherein providing feedback comprises providing visual feedback or aural feedback through a deviation indication device.

20. The method of claim 17, wherein providing feedback comprises restricting movement of the procedure device by providing resistance to motion via the robot arm.

21. A method of performing a procedure, the method comprising:
arranging a distal end of a tracking arm in communication with a reference location at or adjacent to a site or an object received at the site, the reference location being disposed in a three-dimensional space relative to a proximal end of the tracking arm;
contacting a physical point at the site or on the object with a distal end of an end effector of a procedure tool engaged with a distal end of a robot arm, a proximal end of the robot arm being disposed in a known relation to the proximal end of the tracking arm,
determining a location of the distal end of the end effector in the three-dimensional space concurrently with contacting the physical point with the distal end of the end effector,
establishing a trajectory of the end effector, relative to the physical point at the site or on the object, for performing a procedure at the site or on the object;
forming a plan for the procedure to be performed by the end effector at the location of the physical point at the site or on the object, the procedure including a route traversed by the end effector to and from the site or the object and during the procedure performed by the end effector at the location and in the established trajectory at the physical point at the site or on the object; and
performing the procedure at the site or on the object according to the plan, including traversing the route, using the end effector of the procedure tool engaged with the distal end of the robot arm.

22. The method of claim 21, wherein contacting the physical point with the distal end of the end effector of the procedure tool comprises contacting the physical point with the distal end of the end effector, the end effector comprising a drill bit or an abrading bit, of the procedure tool, the procedure tool comprising a drilling device.

23. The method of claim 21, wherein the robot arm comprises a plurality of interconnected arm segments having one or more position sensors engaged therewith, and wherein determining the location of the distal end of the end effector comprises determining the location of the distal end of the end effector, relative to the proximal end of the robot arm, from position data from the one or more position sensors.

24. The method of claim 21, wherein the tracking arm comprises a plurality of interconnected arm segments having one or more position sensors engaged therewith, and wherein the method comprises determining a location of the distal end of the tracking arm, relative to the proximal end thereof, from position data from the one or more position sensors.

25. The method of claim 21, wherein determining the location of the distal end of the end effector in the three-dimensional space comprises determining a location of the site or the object in the three-dimensional space via the end effector, in relation to the reference location, the reference location being physically engaged with the distal end of the tracking arm.

26. The method of claim 21, wherein the distal end of the tracking arm is spaced-apart from the site or the object and includes a detector device engaged therewith, the detector device being arranged in communication with the site or the object, and wherein determining the location of the distal end of the end effector comprises determining a location of the site or the object in the three-dimensional space via the end effector, in relation to the reference location, the reference location being detected by the detector device engaged with the distal end of the tracking arm.

27. The method of claim 21, wherein determining the location of the distal end of the end effector comprises determining the location of the distal end of the end effector, relative to the reference location determined via the tracking arm, in the three-dimensional space.

28. The method of claim 21, wherein performing the procedure comprises performing the procedure at the site or on the object according to the plan, relative to the reference location determined via the tracking arm, including traversing the route, using the end effector of the procedure tool engaged with the distal end of the robot arm.

29. The method of claim 28, comprising adjusting the plan during performance of the procedure in response to movement of the site or the object, as determined via movement of the reference location determined via the tracking arm.

30. The method of claim 21, comprising providing feedback in response to the procedure device deviating from the plan during performance of the procedure, including traversing the route.

31. The method of claim 30, wherein providing feedback comprises directing haptic feedback or tactile feedback through the procedure device.

32. The method of claim 30, wherein providing feedback comprises providing visual feedback or aural feedback through a deviation indication device.

33. The method of claim 30, wherein providing feedback comprises restricting movement of the procedure device by providing resistance to motion via the robot arm.

34. The method of claim 21, wherein forming the plan comprises forming the plan for the procedure to be performed by the end effector at the location of each of a plurality of physical points at the site or on the object, and wherein performing the procedure comprises performing the procedure at each of the physical points at the site or on the object using the end effector in the established trajectory such that the procedure is performed in parallel between the physical points.

35. A method of performing a procedure, the method comprising:
arranging a distal end of a tracking arm in communication with a reference location at or adjacent to a site or an object received at the site, the reference location being disposed in a three-dimensional space relative to a proximal end of the tracking arm;
determining a location of a distal end of an end effector of a procedure tool in the three-dimensional space, the procedure tool being engaged with the distal end of a robot arm, a proximal end of the robot arm being disposed in a known relation to the proximal end of the tracking arm;
establishing a trajectory of the end effector relative to the location of the distal end thereof;
forming a plan for a procedure to be performed by the end effector at a location of a physical point at the site or on the object, the procedure including a route traversed by the end effector to and from the site or the object and during the procedure performed by the end effector at the location and in the established trajectory of the end effector at the physical point at the site or on the object; and
performing the procedure at the site or on the object according to the plan, including traversing the route and orienting the end effector in the established trajectory, using the end effector of the procedure tool engaged with the distal end of the robot arm.

36. The method of claim 35, wherein establishing the trajectory comprises manually orienting the procedure tool such that the end effector is in a three-dimensional angular arrangement relative to the distal end of the end effector, and storing the angular arrangement of the end effector providing the trajectory for use in forming the plan.

37. The method of claim 35, wherein establishing the trajectory comprises determining a three-dimensional angular arrangement of the end effector providing the trajectory; determining a spatial offset of a proximal end of the end effector from the distal end thereof providing the angular arrangement, and storing the spatial offset of the proximal end of the end effector providing the trajectory for use in forming the plan.

38. The method of claim 35, wherein determining the location of the distal end of the end effector comprises determining the location of the distal end of the end effector, the end effector comprising a drill bit or an abrading bit, of the procedure tool, the procedure tool comprising a drilling device.

39. The method of claim 35, wherein determining the location of the distal end of the end effector comprises contacting the physical point with the distal end of the end effector, and concurrently determining a location of the distal end of the end effector in the three-dimensional space.

40. The method of claim 35, wherein the robot arm comprises a plurality of interconnected arm segments having one or more position sensors engaged therewith, and wherein determining the location of the distal end of the end effector comprises determining the location of the distal end of the end effector, relative to the proximal end of the robot arm, from position data from the one or more position sensors.

41. The method of claim 35, wherein the tracking arm comprises a plurality of interconnected arm segments having one or more position sensors engaged therewith, and wherein the method comprises determining a location of the distal end of the tracking arm, relative to the proximal end thereof, from position data from the one or more position sensors.

42. The method of claim 35, wherein determining the location of the distal end of the end effector in the three-dimensional space comprises determining a location of the site or the object in the three-dimensional space via the end effector, in relation to the reference location, the reference location being physically engaged with the distal end of the tracking arm.

43. The method of claim 35, wherein the distal end of the tracking arm is spaced-apart from the site or the object and includes a detector device engaged therewith, the detector device being arranged in communication with the site or the object, and wherein determining the location of the distal end of the end effector comprises determining a location of the site or the object in the three-dimensional space via the end effector, in relation to the reference location, the reference location being detected by the detector device engaged with the distal end of the tracking arm.

44. The method of claim 35, wherein determining the location of the distal end of the end effector comprises determining the location of the distal end of the end effector, relative to the reference location determined via the tracking arm, in the three-dimensional space.

45. The method of claim 35, wherein performing the procedure comprises performing the procedure at the site or on the object according to the plan, relative to the reference location determined via the tracking arm, including traversing the route and orienting the end effector in the established trajectory, using the end effector of the procedure tool engaged with the distal end of the robot arm.

46. The method of claim 45, comprising adjusting the plan during performance of the procedure in response to movement of the site or the object, as determined via movement of the reference location determined via the tracking arm.

47. The method of claim 35, comprising providing feedback in response to the procedure device deviating from the plan during performance of the procedure, including traversing the route and orienting the end effector in the established trajectory.

48. The method of claim 47, wherein providing feedback comprises directing haptic feedback or tactile feedback through the procedure device.

49. The method of claim 47, wherein providing feedback comprises providing visual feedback or aural feedback through a deviation indication device.

50. The method of claim 47, wherein providing feedback comprises restricting movement of the procedure device by providing resistance to motion via the robot arm.

51. The method of claim 35, wherein forming the plan comprises forming the plan for the procedure to be performed by the end effector at the location of each of a plurality of physical points at the site or on the object, and wherein performing the procedure comprises performing the procedure at each of the physical points at the site or on the object using the end effector in the established trajectory such that the procedure is performed in parallel between the physical points.
